(19) United States
(12) Patent Application Publication
(10) Pub. No.: US 2006/0194869 A1
(43) Pub. Date: Aug. 31, 2006

(54) Process for preparing pharmaceutically acceptable salts of duloxetine and intermediates thereof

(76) Inventors: Santiago Ini, Haifa (IL); Anita Liberman, Tel Aviv (IL)

Correspondence Address: KENYON & KENYON LLP, ONE BROADWAY, NEW YORK, NY, 10004, US

(21) Appl. No.: 11/318,365

(22) Filed: Dec. 23, 2005

Related U.S. Application Data
(60) Provisional application No. 60/638,779, filed on Dec. 23, 2004.
(60) Provisional application No. 60/723,492, filed on Oct. 3, 2005.

(51) Int. Cl. A61K 31/381; C07D 333/22

(52) U.S. Cl. 514/438; 549/76

(57) ABSTRACT

Processes for preparing DNT-base, duloxetine alkyl carbamate, duloxetine-base and duloxetine hydrochloride, are provided. Also provided, are processes for converting DNT-base, duloxetine alkyl carbamate and duloxetine-base into pharmaceutically acceptable salts of duloxetine.

### RELATED APPLICATIONS

This application claims benefit of U.S. Provisional Patent Applications Nos. 60/638,779 and 60/723,492, filed Dec. 23, 2004, and Oct. 3, 2005, respectively, the contents of which are incorporated herein in their entirety.

### FIELD OF THE INVENTION

The present invention provides processes for preparing duloxetine intermediates. The present invention also provides processes for converting these duloxetine intermediate into pharmaceutically acceptable salts of duloxetine.

### BACKGROUND OF THE INVENTION

Duloxetine hydrochloride is a dual reuptake inhibitor of the neurotransmitters serotonin and norepinephrine. It is used for the treatment of stress urinary incontinence (SUI), depression, and pain management. Duloxetine hydrochloride has the following chemical structure and name: 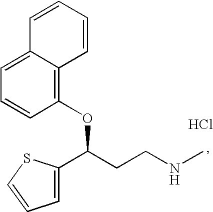
(S)-(+)-N-methyl-3-(1-naphthalenyloxy)-3-(2-thienyl)propanamine hydrochloric acid salt.

Duloxetine base, as well as processes for its preparation, is disclosed in U.S. Pat. No. 5,023,269 (US '269). EP Patent No. 457559 and U.S. Pat. No. 5,491,243 (US '243) and U.S. Pat. No. 6,541,668 provide an improved synthetic route for the preparation of duloxetine base. US '269 describes the preparation of duloxetine base by reacting N,N-Dimethyl-3-(2-thienyl)-3-hydroxypropanamine with fluoronaphtalene (Stage a), followed by demethylation with Phenyl chloroformate or trichloroethyl chloroformate (Stage b) and basic hydrolysis (Stage c) according to the following scheme:

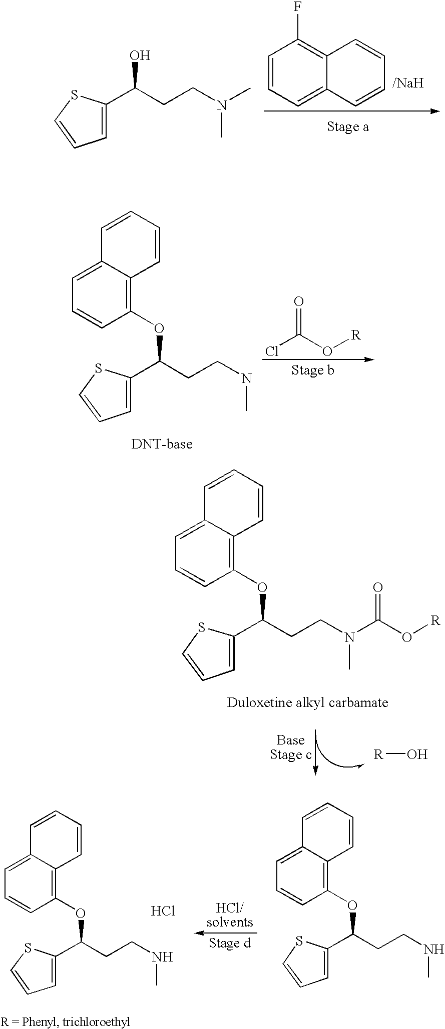

The conversion of duloxetine base to its hydrochloride salt is described in US '243 and in Wheeler, W. J., et al, J. Label. Cpds. Radiopharm, 1995, 36, 312. In both publications, the conversion reactions are performed in ethyl acetate, and the reported yield for this process in the Wheeler, W. J. et. al. publication, is 45%.

EP '559 discloses the conversion of N,N-Dimethyl-3-(1-naphthalenyloxy)-3-(2-thienyl) propanamine oxalate (DNT-Oxal) to N,N-Dimethyl-3-(1-naphthalenyloxy)-3-(2-thienyl) propanamine (DNT-base) with sodium hydroxide.

In US '243, the process described in Stage b is performed in a phenyl chloroformate/diisopropylethylamine system at 55° C., and, in International Patent Application Publication No. WO 04/056795, this stage is performed in the presence of chloroethyl chloroformate at 60° C.

The drawbacks of the process described in the above patents and publication are the use of the phenyl and trichlorinated chloroformates in Stage b, which results in the formation of the very toxic substances, such as phenol and trichloroethanol in Stage c. In addition, these processes require temperatures higher than 55° C.

U.S. Pat. No. 5,023,269 (US '269) and U.S. Pat. No. 5,362,886 (US '886) disclose processes for the reaction of Stage c in which propylene glycol/sodium hydroxide system and dimethylsulfoxide/sodium hydroxide system, respectively are used.

Therefore, there is a need in the art for improved synthetic processes for the preparation of duloxetine intermediates, and ultimately their conversion to duloxetine HCl that reduce the production of toxic byproducts and increase the yields. The present invention provides such processes.

### SUMMARY OF THE INVENTION

In one embodiment, the present invention provides a process for preparing DNT-base, comprising: combining DNT-Oxal, water, an ammonium hydroxide solution, and an organic solvent to obtain an organic solution, containing DNT-base.

Preferably, the DNT-Oxal is (S)-(+) DNT-Oxal and the DNT-base obtained is (S)-DNT-base.

The present invention further provides a process for preparing pharmaceutically acceptable salts of duloxetine comprising: preparing DNT-base as described above, and converting the DNT-base to pharmaceutically acceptable salts of duloxetine.

Preferably, the DNT-base is converted to duloxetine hydrochloride.

Preferably, the DNT-base is (S)-DNT-base and the duloxetine hydrochloride is (S)-(+)-duloxetine hydrochloride.

In another embodiment, the present invention provides a process for preparing duloxetine alkyl carbamate, comprising: dissolving DNT-base in an organic solvent; adding an alkyl chloroformate or a halo alkyl chloroformate (which is not chloroalkyl chloroformate) at a temperature of about 5° C. to less than about 80° C., and recovering the duloxetine alkyl carbamate.

Preferably, the DNT-base is (S)-DNT-base and the duloxetine alkyl carbamate obtained is an (S)-duloxetine alkyl carbamate.

The present invention further provides a process for preparing pharmaceutically acceptable salts of duloxetine comprising: preparing duloxetine alkyl carbamate as described above, and converting the duloxetine alkyl carbamate to pharmaceutically acceptable salts of duloxetine.

Preferably, the duloxetine alkyl carbamate is converted to duloxetine hydrochloride.

Preferably, the duloxetine alkyl carbamate is an (S)-duloxetine alkyl carbamate and the duloxetine hydrochloride is (S)-(+)-duloxetine hydrochloride.

In another embodiment, the present invention provides a process for preparing duloxetine alkyl carbamate, comprising: combining DNT-base, an organic solvent and a proton trap; adding an alkyl chloroformate or a halo alkyl chloroformate (which is not chloroalkyl chloroformate), and recovering the duloxetine alkyl carbamate.

Preferably, the DNT-base is (S)-DNT-base and the duloxetine alkyl carbamate obtained is an (S)-duloxetine alkyl carbamate.

The present invention further provides a process for preparing pharmaceutically acceptable salts of duloxetine comprising: preparing duloxetine alkyl carbamate as described above, and converting the duloxetine alkyl carbamate to pharmaceutically acceptable salts of duloxetine.

Preferably, the duloxetine alkyl carbamate is converted to duloxetine hydrochloride.

Preferably, the duloxetine alkyl carbamate is an (S)-duloxetine alkyl carbamate and the duloxetine hydrochloride is (S)-(+)-duloxetine hydrochloride.

In another embodiment, the present invention provides a process for preparing duloxetine-base comprising: combining duloxetine alkyl carbamate and an organic solvent selected from the group consisting of an aliphatic alcohol, ether and an aromatic hydrocarbon, with a base selected from the group consisting of KOH and NaOH.

Preferably, the duloxetine alkyl carbamate is an (S)-duloxetine alkyl carbamate and the duloxetine-base obtained is (S)-duloxetine-base.

The present invention further provides a process for preparing pharmaceutically acceptable salts of duloxetine comprising: preparing duloxetine-base as described above, and converting the duloxetine-base to pharmaceutically acceptable salts of duloxetine.

Preferably, the duloxetine-base is converted to duloxetine hydrochloride.

Preferably, the duloxetine-base is (S)-duloxetine-base and the duloxetine hydrochloride is (S)-(+)-duloxetine hydrochloride.

In another embodiment, the present invention provides a process for preparing duloxetine hydrochloride comprising: combining duloxetine-base and a solvent selected from the group consisting of water, an aromatic hydrocarbon, a C₁₋₄ ester, which is not ethyl acetate, a C₂₋₈ ether, a C₁₋₈ alcohol, acetonitrile and a ketone; adding hydrochloric acid in an amount sufficient to provide a pH of about 1 to about 5 to obtain duloxetine hydrochloride, and recovering duloxetine hydrochloride.

Preferably, the duloxetine-base is (S)-duloxetine-base and the duloxetine hydrochloride obtained is (S)-(+) duloxetine hydrochloride.

In another embodiment, the present invention provides a process for preparing duloxetine hydrochloride comprising:
a) combining DNT-Oxal, water, an ammonium hydroxide solution, and an organic solvent, to obtain an organic solution, containing DNT-base;
b) dissolving the DNT-base in a second organic solvent;
c) adding an alkyl chloroformate or a halo alkyl chloroformate (which is not chloroalkyl chloroformate) at a temperature of about 5° C. to less than about 80° C.;
d) recovering the duloxetine alkyl carbamate;
e) combining the duloxetine alkyl carbamate and an organic solvent selected from the group consisting of an aliphatic alcohol, ether and an aromatic hydrocarbon, with an alkaline metal base;
f) recovering duloxetine-base;
g) combining the duloxetine-base and a solvent selected from the group consisting of an aromatic hydrocarbon, a C₁₋₄ ester, which is not ethyl acetate, a C₂₋₈ ether, a C₁₋₈ alcohol, acetonitrile and a ketone;
h) adding hydrochloric acid in an amount sufficient to provide a pH of about 1 to about 5;
i) maintaining the reaction mixture to obtain a solid residue; and
j) recovering duloxetine hydrochloride.

In another embodiment, the present invention provides a process for preparing duloxetine hydrochloride comprising:
a) combining DNT-Oxal, water, an ammonium hydroxide solution, and an organic solvent to obtain an organic solution, containing DNT-base;
b) combining the DNT-base, a second organic solvent and a proton trap;
c) adding an alkyl chloroformate or a halo alkyl chloroformate (which is not chloroalkyl chloroformate);
d) recovering the duloxetine alkyl carbamate;
e) combining the duloxetine alkyl carbamate and an organic solvent selected from the group consisting of an aliphatic alcohol, ether and an aromatic hydrocarbon, with an alkaline metal base;
f) recovering duloxetine-base;
g) combining the duloxetine-base and a solvent selected from the group consisting of an aromatic hydrocarbon, a C₁₋₄ ester, which is not ethyl acetate, a C₂₋₈ ether, a C₁₋₈ alcohol, acetonitrile and a ketone;
h) adding hydrochloric acid in an amount sufficient to provide a pH of about 1 to about 5;
i) maintaining the reaction mixture to obtain a solid residue; and
j) recovering duloxetine hydrochloride.

### DETAILED DESCRIPTION OF THE INVENTION

As used herein, the term DNT-Oxal refers to N,N-Dimethyl-3-(1-naphthalenyloxy)-3-(2-thienyl) propanamine oxalate, and the term DNT-base refers to N,N-Dimethyl-3-(1-naphthalenyloxy)-3-(2-thienyl) propanamine.

The present invention provides processes for preparing DNT-base, converting the DNT-base into duloxetine carbamate intermediates, and the conversion of the duloxetine carbamate intermediates into duloxetine-base and duloxetine hydrochloride.

In one embodiment, the present invention provides a process for preparing DNT-base, comprising: combining DNT-Oxal, water, an ammonium hydroxide solution, and an organic solvent to obtain an organic solution, containing DNT-base.

The DNT-Oxal used in the above process and the DNT-base obtained, may be either racemic or enantiomeric.

Preferably, the DNT-Oxal is (S)-(+) DNT-Oxal and the DNT-base obtained is (S)-DNT-base.

Preferably, the temperature in which the DNT-Oxal is combined with water, an ammonium hydroxide solution, and an organic solvent, is about room temperature, i.e., from about 18° to about 30° C., more preferably, from about 20 to about 25° C.

Preferably, the organic solvent is selected from the group consisting of aromatic hydrocarbons, C₄₋₈ alcohols, ketones, esters and ethers. More preferably the organic solvent is an alcohol such as butanol or an aromatic hydrocarbon such as benzene, toluene, xylene, ethyl benzene, propyl benzene, or an ether such as diethyl ether, dipropyl ether, dibutyl ether. Most preferably the organic solvent is toluene.

The present invention further provides a process for preparing pharmaceutically acceptable salts of duloxetine comprising: preparing DNT-base as described above, and converting the DNT-base to pharmaceutically acceptable salts of duloxetine.

Preferably, the DNT-base is converted to duloxetine hydrochloride.

Preferably, the DNT-base is (S)-DNT-base and the duloxetine hydrochloride is (S)-(+)-duloxetine hydrochloride.

The preparation of the DNT-base is performed using ammonium hydroxide, which prevents undesirable precipitation and formation of by-products, such as observed in prior art, when using Sodium Hydroxide.

In another embodiment, the present invention provides a process for preparing duloxetine alkyl carbamate, comprising: dissolving DNT-base in an organic solvent; adding an alkyl chloroformate or a halo alkyl chloroformate (which is not chloroalkyl chloroformate) at a temperature of about 5° C. to less than about 80° C., and recovering the duloxetine alkyl carbamate.

The DNT-base used in the above process and the duloxetine alkyl carbamate obtained, may be either racemic or enantiomeric.

Preferably, the DNT-base is (S)-DNT-base and the duloxetine alkyl carbamate obtained is an (S)-duloxetine alkyl carbamate.

Preferably, the alkyl residue of the carbamate is a C₁₋₈ branched or unbrunched alkyl, such as ethyl or isobutyl. Most preferably, the alkyl is ethyl.

Preferably, the organic solvent is selected from the group consisting of C₄₋₈ substituted or unsubstituted, aliphatic or aromatic hydrocarbons, C₁₋₆ linear or branched esters and acetonitrile.

A preferred aliphatic hydrocarbon is heptane. Preferred aromatic hydrocarbons are benzene, toluene and xylene. A most preferred aromatic hydrocarbon is toluene. Preferred C₁₋₆ esters are methyl acetate, ethyl acetate, n-propyl acetate, i-propyl acetate, n-butyl acetate, s-butyl acetate, i-butyl acetate, t-butyl acetate, benzyl acetate and phenyl acetate. A most preferred C₁₋₆ ester is ethyl acetate.

Preferably, the alkyl chloroformate is added at a temperature of about 50° C.

Preferably, any water present in the reaction mixture is removed. Removal of water is performed by any means known in the art, such as azeotropic distillation at high temperatures, or drying under any suitable drying agent

The present invention further provides a process for preparing pharmaceutically acceptable salts of duloxetine comprising: preparing duloxetine alkyl carbamate as described above, and converting the duloxetine alkyl carbamate to pharmaceutically acceptable salts of duloxetine.

Preferably, the duloxetine alkyl carbamate is converted to duloxetine hydrochloride.

Preferably, the duloxetine alkyl carbamate is an (S)-duloxetine alkyl carbamate and the duloxetine hydrochloride is (S)-(+)-duloxetine hydrochloride.

In another embodiment, the present invention provides a process for preparing duloxetine alkyl carbamate, comprising: combining DNT-base, an organic solvent and a proton trap; adding an alkyl chloroformate or a halo alkyl chloroformate (which is not chloroalkyl chloroformate), and recovering the duloxetine alkyl carbamate.

The DNT-base used in the above process and the duloxetine alkyl carbamate obtained, may be either racemic or enantiomeric.

Preferably, the DNT-base is (S)-DNT-base and the duloxetine alkyl carbamate obtained is an (S)-duloxetine alkyl carbamate.

Preferably, the alkyl residue of the carbamate, as well as the organic solvent, are as described above.

The proton trap is a base which forms a salt with an acid, present in the reaction, without interfering in the reaction. Preferably, the proton trap is selected from the group consisting of a C₃-C₈ trialkyl amine, bicarbonates, Na₂CO₃ and K₂CO₃. More preferably, the proton trap is selected from the group consisting of diisopropyl ethyl amine, tributyl amine and K₂CO₃. Most preferably, the proton trap is K₂CO₃.

Preferably, any water present in the reaction mixture is removed. Removal of water is performed as described above.

The duloxetine carbamates prepared according to any one of the above methods may be recovered by any method known in the art, such as separating the phases, and concentrating the organic phase until a dry residue is formed. Prior to separation, the carbamate may be washed in order to remove inorganic or organic impurities. To further purify the carbamate intermediate, it may be washed, in addition to water, with weak bases, such as NH₄OH and aqueous acids solutions, such as aqueous HCl.

The present invention further provides a process for preparing pharmaceutically acceptable salts of duloxetine comprising: preparing duloxetine alkyl carbamate as described above, and converting the duloxetine alkyl carbamate to pharmaceutically acceptable salts duloxetine.

Preferably, the duloxetine alkyl carbamate is converted to duloxetine hydrochloride.

Preferably, the duloxetine alkyl carbamate is an (S)-duloxetine alkyl carbamate and the duloxetine hydrochloride is (S)-(+)-duloxetine hydrochloride

The preparation of the carbamate intermediates is performed using an alkyl chloroformate, such that, during hydrolysis of the carbamate to duloxetine, the alcohol byproduct is an alkyl alcohol. Disposal of the alkyl alcohol is much more convenient and environmentally safe, when compared to the alcohols, such as phenol, produced in prior art processes.

In another embodiment, the present invention provides a process for preparing duloxetine-base comprising: combining duloxetine alkyl carbamate and an organic solvent selected from the group consisting of an aliphatic alcohol, ether and an aromatic hydrocarbon, with an alkaline metal base, and recovering duloxetine-base.

The duloxetine alkyl carbamate used in the above process and the duloxetine-base obtained, may be either racemic or enantiomeric.

Preferably, the duloxetine alkyl carbamate is an (S)-duloxetine alkyl carbamate and the duloxetine-base obtained is (S)-duloxetine-base.

Preferably, the organic solvent is selected from the group consisting of EtOH, IPA, Ethylene Glycol Diethyl Ether, propylene glycol methyl ether, DMSO and toluene.

Preferably, the organic solvent is toluene.

Preferably, the base is KOH.

Preferably, after combining the duloxetine alkyl carbamate and an organic solvent with a base, the reaction mixture is maintained at a temperature of from about 60° C. to about the reflux temperature of the solvent, for about 1 to 4 hours.

The present invention further provides a process for preparing pharmaceutically acceptable salts of duloxetine comprising: preparing duloxetine-base as described above, and converting the duloxetine-base to pharmaceutically acceptable salts of duloxetine.

Preferably, the duloxetine-base is converted to duloxetine hydrochloride.

Preferably, the duloxetine-base is (S)-duloxetine-base and the duloxetine hydrochloride is (S)-(+)-duloxetine hydrochloride.

The preparation of the duloxetine-base is performed using a solvent/base pair of toluene/KOH, which increases the yield, such as observed in prior art, when using propylene glycol/sodium hydroxide system and dimethylsulfoxide/sodium hydroxide system. Also, the use of toluene/KOH allows the preparation of duloxetine-base directly from the reaction mixture obtained when making the duloxetine alkyl carbamate, using the same solvent used in the duloxetine alkyl carbamate preparation, and thus, having an industrial and ecological adventages.

In another embodiment, the present invention provides a process for preparing duloxetine hydrochloride comprising: combining duloxetine-base and a solvent selected from the group consisting of water, an aromatic hydrocarbon, a C₁₋₄ ester, which is not ethyl acetate, a C₂₋₈ ether, a C₁₋₈ alcohol, acetonitrile and a ketone; adding hydrochloric acid in an amount sufficient to provide a pH of about 1 to about 5 to obtain duloxetine hydrochloride, and recovering duloxetine hydrochloride.

The duloxetine-base used in the above process and the duloxetine hydrochloride obtained, may be either racemic or enantiomeric.

Preferably, the duloxetine-base is (S)-duloxetine-base and the duloxetine hydrochloride obtained is (S)-(+) duloxetine hydrochloride.

Preferably, the solvent is selected from the group consisting of water, toluene, isopropyl alcohol, methanol, acetone, methyl ethyl ketone, diethyl ether, MTBE or mixtures thereof. Most preferably, the solvent is acetone.

A one-pot reaction is also feasible, wherein, instead of a solvent, hydrochloric acid is combined with duloxetine-base.

The solvents used in the above process produce duloxetine hydrochloride in high yield.

In another embodiment, the present invention provides a process for preparing duloxetine hydrochloride comprising:
a) combining DNT-Oxal, water, an ammonium hydroxide solution, and an organic solvent, to obtain an organic solution, containing DNT-base;
b) dissolving the DNT-base in a second organic solvent;
c) adding an alkyl chloroformate or a halo alkyl chloroformate (which is not chloroalkyl chloroformate) at a temperature of about 5° C. to less than about 80° C.;
d) recovering the duloxetine alkyl carbamate;
e) combining the duloxetine alkyl carbamate and an organic solvent selected from the group consisting of an aliphatic alcohol such as EtOH, IPA or an ether such as Ethylene Glycol Diethyl Ether, propylene glycol methyl ether, DMSO or an aromatic solvent, such as toluene with an alkaline metal base;
f) recovering duloxetine-base;
g) combining the duloxetine-base and a solvent selected from the group consisting of an aromatic hydrocarbon, a C₁₋₄ ester, which is not ethyl acetate, a C₂₋₈ ether, a C₁₋₈ alcohol, acetonitrile and a ketone;
h) adding hydrochloric acid in an amount sufficient to provide a pH of about 1 to about 5;
i) maintaining the reaction mixture to obtain a solid residue; and
j) recovering duloxetine hydrochloride.

In another embodiment, the present invention provides a process for preparing duloxetine hydrochloride comprising:
a) combining DNT-Oxal, water, an ammonium hydroxide solution, and an organic solvent to obtain an organic solution, containing DNT-base;
b) combining the DNT-base, a second organic solvent and a proton trap;
c) adding an alkyl chloroformate or a halo alkyl chloroformate (which is not chloroalkyl chloroformate);
d) recovering the duloxetine alkyl carbamate;
e) combining the duloxetine alkyl carbamate and an organic solvent selected from the group consisting of an aliphatic alcohol such as EtOH, IPA or an ether such as Ethylene Glycol Diethyl Ether, propylene glycol methyl ether, DMSO or an aromatic solvent, such as toluene with an alkaline metal base;
f) recovering duloxetine-base;
g) combining the duloxetine-base and a solvent selected from the group consisting of an aromatic hydrocarbon, a C₁₋₄ ester, which is not ethyl acetate, a C₂₋₈ ether, a C₁₋₈ alcohol, acetonitrile and a ketone;
h) adding hydrochloric acid in an amount sufficient to provide a pH of about 1 to about 5;
i) maintaining the reaction mixture to obtain a solid residue; and
j) recovering duloxetine hydrochloride.

Having described the invention with reference to certain preferred embodiments, other embodiments will become apparent to one skilled in the art from consideration of the specification. The invention is further defined by reference to the following examples describing in detail the preparation of the composition and methods of use of the invention. It will be apparent to those skilled in the art that many modifications, both to materials and methods, may be practiced without departing from the scope of the invention.

### EXAMPLES

Preparation of (S)-Duloxetine Ethyl Carbamate

### Example 1

A 100 ml three necked flask, equipped with mechanical stirrer, thermometer, dean stark, and condenser, was charged with 5 g of (S)-DNT-base and 25 ml of toluene. The clear solution was heated, and an azeotropic distillation was performed for about 30 to about 60 minutes. After cooling to room temperature, 4.6 ml of ethyl chloroformate were added during over a period of 1 to 2 hours, and the reaction mixture was stirred at room temperature over night.

Diluted NH₄OH was added to the reaction mixture, which was stirred for an additional 30 minutes. After phase separation, the organic phase was washed with water (3×20 ml), dried over Na₂SO₄, filtered, and concentrated to dryness to give 5.2 g of a brownish oil. (88% chemical yield).

### Example 2

A 100 ml three necked flask, equipped with mechanical stirrer, thermometer, dean stark, and condenser, was charged with 4 g of (S)-DNT-base and 20 ml of toluene. The clear solution was heated, and an azeotropic distillation was performed for about 30 to about 60 minutes. After cooling to 60° C., 3.7 ml of ethyl chloroformate were added over a period of 1 hour, and the reaction mixture was stirred at the same temperature for an additional 4.5 hours.

The resulting reaction mixture was washed with diluted HCl, water, diluted NH₄OH, and water again. After phase separation, the organic solution was dried over Na₂SO₄, filtered, and concentrated to dryness to give 3.59 g of a brownish oil. (76% chemical yield)

### Example 3

A 100 ml three necked flask, equipped with mechanical stirrer, thermometer, dean stark, and condenser, was charged with 4 g (S)-DNT-base and 20 ml toluene. The clear solution was heated, and an azeotropic distillation was performed for about 30 to about 60 minutes. After cooling, 3.4 ml of diisopropyl ethyl amine were added, and the reaction mixture was heated to 60° C. Then, 3.7 ml of ethyl chloroformate were added over a period of 1 hour, and the reaction mixture stirred at the same temperature for an additional 1.5 hours.

The resulting reaction mixture was washed with diluted HCl and water, and diluted with NH₄OH and water again. After phase separation, the organic solution was dried over Na₂SO₄, filtered, and concentrated to dryness to give 4.17 g of a brownish oil. (88% yield).

### Example 4

A 100 ml three necked flask, equipped with mechanical stirrer, thermometer, and condenser, was charged with 4 g (S)-DNT-base, 20 ml of n-heptane, and 3.4 ml of diisopropyl ethyl amine. The mixture was heated to 60° C. Then, 3.7 ml of ethyl chloroformate were added over a period of 1 hour, and the reaction mixture was stirred at the same temperature for an additional 2.5 hours.

### Example 5

A 100 ml three necked flask, equipped with mechanical stirrer, thermometer, and condenser, was charged with 6 g (S)-DNT-base, 30 ml of acetonitrile, and 2 g of K₂CO₃. The mixture was heated to 60° C. Then, 6.3 g of ethyl chloroformate were added over a period of 1 hour, and the reaction mixture stirred at the same temperature for an additional hour. The resulting reaction mixture was washed with water, and diluted with 60 ml EtOAc, followed by washing with diluted HCl and brine. The organic solution was dried over Na₂SO₄, filtered, and concentrated to dryness to give 2.75 g of brownish oil. (38.67% yield).

Preparation of (S)-Duloxetine Isobutyl Carbamate

### Example 6

A 100 ml three necked flask, equipped with mechanical stirrer, thermometer, dean stark, and condenser, was charged with 6 g (S)-DNT-base, 2.01 g of K₂CO₃ and 30 ml toluene. The mixture was heated, and an azeotropic distillation was performed for about 30 to about 60 minutes. After cooling to 60° C., 3.7 ml of isobutyl chloroformate were added over a period of ½ hour, and the reaction mixture was stirred at the same temperature for an additional 2.5 hours.

The resulting reaction mixture was washed with diluted HCl, water, diluted NaHCO₃, NH₄OH, and water again. After phase separation, the organic solution was dried over Na₂SO₄, filtered, and concentrated to dryness to give 5.71 g of a brownish oil. (74.54% yield).

Preparation of (S)-Duloxetine Base

### Example 7

A 100 ml three necked flask equipped, with mechanical stirrer, thermometer, and condenser, was charged with 2.5 g (S)-duloxetine ethyl carbamate and 20 ml toluene. The mixture was stirred, and 4.8 g of KOH were added in portions, followed by reflux for about 3 hours.

After cooling, 30 ml of water, followed by 20 ml of toluene, were added, and the resulting organic phase was washed with water (3×20 ml), dried over Na₂SO₄, filtered and concentrated to dryness to give 1.70 g of an oily product. (85.31% yield).

Preparation of (S)-(+)-Duloxetine Hydrochloric

### Example 8

To a mixture of 2 g of (S)-duloxetine in 15 ml water was slowly added a 32 percent solution of hydrochloric acid until the pH reached 3 to 4. The mixture, was stirred until the yellow oil turned into a white solid. The resulting solid was filtered, washed with water, and dried in a vacuum oven to give 1.30 g of (S)-(+)-duloxetine hydrochloride as a white solid, having a purity of 99.60 percent purity, based on HPLC area percent and 57.94% yield.

### Example 9

To a solution of 1.9 g of (S)-duloxetine in 20 ml toluene was slowly added 2.4 ml of a 10 percent solution of hydrochloric acid or until a pH of 3 to 4 was obtained. The mixture was stirred for an hour, until the yellow oil turned into a solid. The resulting solid was filtered, washed with 20 ml of toluene, and dried in a vacuum oven to give 1.20 g of (S)-(+)-duloxetine hydrochloride. (56.34% yield).

### Example 10

To a solution of 2 g of (S)-duloxetine in 20 ml toluene was slowly added 7 ml of saturated HCl/toluene or until a pH of 3 was obtained. The mixture was stirred until a white solid was formed. The resulting solid was filtered, washed with toluene, and dried in a vacuum oven to give 1.30 g of (S)-(+)-duloxetine hydrochloride. (57.94% yield).

### Example 11

To a solution of 1.95 g of (S)-duloxetine in 20 ml isopropyl alcohol was slowly added 3 ml of a saturated HCl/isopropyl alcohol solution or until a pH of 1 was obtained. The mixture was stirred until a white solid was formed. The resulting solid was filtered, washed with isopropyl alcohol, and dried in a vacuum oven to give 1.35 g of (S)-(+)-duloxetine hydrochloride. (61.64% yield).

### Example 12

To a solution of 2 g of (S)-duloxetine in 20 ml acetone was slowly added 2 ml of a saturated HCl/acetone solution or until a pH of 1 was obtained. The mixture was stirred until a white solid was formed. The resulting solid was filtered, washed with acetone, and dried in a vacuum oven to give 1.24 g of (S)-(+)-duloxetine hydrochloride. (55.21% yield).

### Example 13

To a solution of 2 g of (S)-duloxetine in 20 ml diethyl ether was slowly added 2 ml of a saturated HCl/diethyl ether solution or until a pH of 2 was obtained. The mixture was stirred until a solid was formed. The resulting solid was filtered, washed with diethyl ether, and dried in a vacuum oven to give 1.82 g of (S)-(+)-duloxetine hydrochloride. (81.10% yield).

### Example 14

To a solution of 1 g of (S)-duloxetine in 10 ml isopropyl alcohol was slowly added 0.32 ml of a 37 percent hydrochloric acid solution. The mixture was stirred until a white solid formed. The resulting solid was filtered out, and dried in a vacuum oven to give 0.98 g of (S)-(+)-duloxetine hydrochloride. (87.5% yield).

### Example 15

To a solution of 1 g of (S)-duloxetine in 10 ml MTBE was slowly added 0.32 ml of a 37 percent hydrochloric acid solution. The mixture was stirred until a solid formed. The resulting solid was filtered, and then dried in a vacuum oven to give 1.03 g of (S)-(+)-duloxetine hydrochloride. (91.96% yield).

### Example 16

To a solution of 1 g of (S)-duloxetine in 10 ml methanol was slowly added 0.32 ml of a 37 percent hydrochloric acid solution. The mixture was stirred for at least an 1 hour, and the product was precipitated out by the addition of ether. The resulting off white solid was filtered, and dried in a vacuum oven to give 0.70 g of (S)-(+)-duloxetine hydrochloride. (62.50% yield).

### Example 17

To a solution of 1 g of (S)-duloxetine in 10 ml MEK was slowly added 0.32 ml of a 37 percent hydrochloric acid solution. The mixture was stirred until a solid formed. The resulting solid was filtered, and dried in a vacuum oven to give 0.50 g of (S)-(+)-duloxetine hydrochloride. (94.64% yield).

Preparation of (S)-DNT-Base

### Example 18

A 2 liter reactor, equipped with a mechanical stirrer, was charged with a mixture of 100 g of (S)-(+)-DNT-Oxal, 600 ml of water, 96 ml of a 22 percent ammonium hydroxide solution, and 1 liter of toluene. The mixture was stirred at 25° C. for 20 to 30 minutes, and the organic phase was separated and washed three times with 300 ml of water, providing a toluene solution of (S)-DNT-base, which was used in Example 19 without evaporation.

Preparation of (S)-Duloxetine Ethyl Carbamate

### Example 19

A 1 liter reactor, equipped with a mechanical stirrer, thermometer, dean stark, and condenser, was charged with (S)-DNT-base obtained in Example 18 dissolved in 1020 ml of toluene and 13 g of K₂CO₃. The mixture was heated, and an azeotropic distillation of 284 ml of the mixture was performed. After cooling to 50° C., 47.46 ml of ethyl chloroformate were added over a period of a half hour, and the reaction mixture was stirred at the same temperature for an additional 2 hours. After cooling to room temperature, the reaction mixture was washed with 230 ml of water, 130 ml of a 5 percent HCl solution, 130 ml of water, 130 ml of a 5 percent NaHCO₃ solution, and 130 ml of water. The resulting toluene solution of (S)-duloxetine ethyl carbamate was used in Example 20 without evaporation.

Preparation of (S)-Duloxetine Base

### Example 20

A 1 liter reactor, equipped with mechanical stirrer, thermometer, and condenser, was charged with the solution of (S)-duloxetine ethyl carbamate in toluene prepared in Example 19. The mixture was heated, and an azeotropic distillation of 268 ml was performed. After cooling to 60° C., 82.18 g of an 85 percent KOH solution were added and the mixture was heated to 94° C. for about 4 hours. After cooling to 60° C., 270 ml of water were added, and the resulting organic phase was washed three times with 270 ml of water, and treated with 4.6 g of charcoal (SX1) for 15 minutes, filtrated through a hyperflow bed, and washed with 60 ml of toluene. The solution was distillated at 30° to 40° C. under a vacuum of 20 to 30 mmHg until a volume of about 1 to 2 volumes of toluene was obtained. The resulting toluene solution of (S)-duloxetine base was used in Example 21.

Preparation of (S)-(+)-Duloxetine Hydrochloric

### Example 21

A 1 liter reactor, equipped with mechanical stirrer, thermometer, and condenser, was charged with the solution of (S)-duloxetine-base in toluene prepared in Example 20. After cooling to room temperature, 670 ml of acetone were added, and the solution was heated to 30° C. Hydrogen chloride gas was bubbled into the solution until the pH the mixture was adjusted to 3 to 5, and the mixture was stirred at the same temperature for 1 hour. After cooling to room temperature, the resulting solid was filtrated out and washed three times with 100 ml of acetone. After drying in a vacuum oven at 45° C. for 15 hours, 47.5 g of (S)-(+)-duloxetine hydrochloride were obtained as an off white powder having a purity of 99.42%, based on HPLC area percent with an overall yield of 56.66%.

While it is apparent that the invention disclosed herein is well calculated to fulfill the objects stated above, it will be appreciated that numerous modifications and embodiments may be devised by those skilled in the art. Therefore, it is intended that the appended claims cover all such modifications and embodiments as falling within the true spirit and scope of the present invention.

What is claimed is:

1. A process for preparing DNT-base, comprising: combining DNT-Oxal, water, an ammonium hydroxide solution, and an organic solvent to obtain an organic solution, containing DNT-base.

2. The process of claim 1, wherein the DNT-Oxal is (S)-(+) DNT-Oxal and the DNT-base obtained is (S)-DNT-base.

3. The process of claim 1, wherein the process is performed at a temperature of from about 18° C. to about 30° C.

4. The process of claim 3, wherein the process is performed at a temperature of from about 20° C. to about 25° C.

5. The process of claim 1, wherein the organic solvent is selected from the group consisting of aromatic hydrocarbons, C₄₋₈ alcohols, ketones, esters and ethers.

6. The process of claim 5, wherein the organic solvent is selected from the group consisting of butanol, benzene, toluene, xylene, ethyl benzene, propyl benzene, diethyl ether, dipropyl ether and dibutyl ether.

7. The process of claim 6, wherein the organic solvent is selected from the group consisting of butanol and toluene.

8. A process for preparing pharmaceutically acceptable salts of duloxetine comprising:
a. preparing DNT-base according to claim 1; and
b. converting the DNT-base to pharmaceutically acceptable salts of duloxetine.

9. The process of claim 8, wherein, in step b), the DNT-base is converted to duloxetine hydrochloride.

10. A process for preparing duloxetine alkyl carbamate, comprising:
a. dissolving DNT-base in an organic solvent;
b. adding an alkyl chloroformate or a halo alkyl chloroformate (which is not chloroalkyl chloroformate) at a temperature of about 5° C. to less than about 80° C.; and
c. recovering duloxetine alkyl carbamate.

11. The process of claim 10, wherein the DNT-base is (S)-DNT-base and the duloxetine alkyl carbamate obtained is an (S)-duloxetine alkyl carbamate.

12. The process of claim 10, wherein the alkyl residue of the carbamate is a C₁₋₈ branched or unbranched alkyl selected from the group consisting of ethyl and isobutyl.

13. The process of claim 12, wherein the alkyl residue is ethyl.

14. The process of claim 10, wherein the organic solvent is selected from the group consisting of C₄₋₈ substituted or unsubstituted, aliphatic or aromatic hydrocarbons, C₁₋₆ linear or branched esters and acetonitrile.

15. The process of claim 14, wherein the organic solvent is selected from the group consisting of heptane, benzene, toluene, xylene, methyl acetate, ethyl acetate, n-propyl acetate, i-propyl acetate, n-butyl acetate, s-butyl acetate, i-butyl acetate, t-butyl acetate, benzyl acetate and phenyl acetate.

16. The process of claim 15, wherein the organic solvent is selected from the group consisting of toluene and ethyl acetate.

17. The process of claim 10, wherein the alkyl chloroformate is added at a temperature of about 50° C.

18. The process of claim 10, wherein any water present in the reaction mixture is removed using azeotropic distillation at high temperatures or drying under any suitable drying agent.

19. A process for preparing pharmaceutically acceptable salts of duloxetine comprising:
a. preparing duloxetine alkyl carbamate according to claim 10; and
b. converting the duloxetine alkyl carbamate to pharmaceutically acceptable salts of duloxetine.

20. The process of claim 19, wherein, in step b), the duloxetine alkyl carbamate is converted to duloxetine hydrochloride.

21. A process for preparing duloxetine alkyl carbamate, comprising:
a. combining DNT-base, an organic solvent and a proton trap;
b. adding an alkyl chloroformate or a halo alkyl chloroformate (which is not chloroalkyl chloroformate); and
c. recovering the duloxetine alkyl carbamate.

22. The process of claim 21, wherein the DNT-base is (S)-DNT-base and the duloxetine alkyl carbamate obtained is an (S)-duloxetine alkyl carbamate.

23. The process of claim 21, wherein the alkyl residue of the carbamate is a C₁₋₈ branched or unbranched alkyl selected from the group consisting of ethyl and isobutyl.

24. The process of claim 23, wherein the alkyl residue is ethyl.

25. The process of claim 21, wherein the organic solvent is selected from the group consisting of C₄₋₈ substituted or unsubstituted, aliphatic or aromatic hydrocarbons, C₁₋₆ linear or branched esters and acetonitrile.

26. The process of claim 25, wherein the organic solvent is selected from the group consisting of heptane, benzene, toluene, xylene, methyl acetate, ethyl acetate, n-propyl acetate, i-propyl acetate, n-butyl acetate, s-butyl acetate, i-butyl acetate, t-butyl acetate, benzyl acetate and phenyl acetate.

27. The process of claim 26, wherein the organic solvent is selected from the group consisting of toluene and ethyl acetate.

28. The process of claim 21, wherein the proton trap is selected from the group consisting of C₃-C₈ trialkyl amine, bicarbonates, Na₂CO₃ and K₂CO₃.

29. The process of claim 28, wherein the proton trap is selected from the group consisting of diisopropyl ethyl amine, tributyl amine and K₂CO₃.

30. The process of claim 29, wherein the proton trap is K₂CO₃.

31. The process of claim 21, wherein any water present in the reaction mixture is removed using azeotropic distillation at high temperatures or drying under any suitable drying agent.

32. A process for preparing pharmaceutically acceptable salts of duloxetine comprising:
a. preparing duloxetine alkyl carbamate according to claim 21; and
b. converting the duloxetine alkyl carbamate to pharmaceutically acceptable salts of duloxetine.

33. The process of claim 32, wherein, in step b), the duloxetine alkyl carbamate is converted to duloxetine hydrochloride

34. A process for preparing duloxetine-base comprising:
a. combining duloxetine alkyl carbamate and an organic solvent selected from the group consisting of an aliphatic alcohol, ether and an aromatic hydrocarbon, with an alkaline metal; and
b. recovering duloxetine-base.

35. The process of claim 34, wherein the duloxetine alkyl carbamate is an (S)-duloxetine alkyl carbamate and the duloxetine-base obtained is (S)-duloxetine-base.

36. The process of claim 34, wherein the organic solvent is selected from the group consisting of EtOH, IPA, Ethylene Glycol Diethyl Ether, propylene glycol methyl ether, DMSO and toluene.

37. The process of claim 36, wherein the organic solvent is toluene.

38. The process of claim 34, wherein the base is KOH.

39. The process of claim 34, wherein after step a) the reaction mixture is maintained at a temperature of from about 60° C. to about the reflux temperature of the solvent, for about 1 to 4 hours.

40. A process for preparing pharmaceutically acceptable salts of duloxetine comprising:
a. preparing duloxetine-base according to claim 34; and
b. converting the duloxetine-base to pharmaceutically acceptable salts of duloxetine.

41. The process of claim 40, wherein, in step b), the duloxetine-base is converted to duloxetine hydrochloride

42. The process of claim 41, wherein the converting of duloxetine-base to duloxetine hydrochloride comprises adding hydrochloric acid in an amount sufficient to provide a pH of about 1 to about 5 to obtain duloxetine hydrochloride.

43. A process for preparing duloxetine hydrochloride comprising:
a. combining duloxetine-base and a solvent selected from the group consisting of water, an aromatic hydrocarbon, a C₁₋₄ ester, which is not ethyl acetate, a C₂₋₈ ether, a C₁₋₈ alcohol, acetonitrile and a ketone;
b. adding hydrochloric acid in an amount sufficient to provide a pH of about 1 to about 5 to obtain duloxetine hydrochloride; and
c. recovering duloxetine hydrochloride.

44. The process of claim 43, wherein the duloxetine-base is (S)-duloxetine-base and the duloxetine hydrochloride obtained is (S)-(+) duloxetine hydrochloride.

45. The process of claim 43, wherein the solvent is selected from the group consisting of water, toluene, isopropyl alcohol, methanol, acetone, methyl ethyl ketone, diethyl ether, MTBE or mixtures thereof.

46. The process of claim 45, wherein the solvent is acetone.

47. A process for preparing duloxetine hydrochloride comprising:
a. combining DNT-Oxal, water, an ammonium hydroxide solution, and an organic solvent, to obtain an organic solution, containing DNT-base;
b. dissolving DNT-base in a second organic solvent;
c. adding an alkyl chloroformate or a halo alkyl chloroformate (which is not chloroalkyl chloroformate) at a temperature of about 5° C. to less than about 80° C.;
d. recovering the duloxetine alkyl carbamate;
e. combining the duloxetine alkyl carbamate and an organic solvent selected from the group consisting of an aliphatic alcohol, ether and an aromatic hydrocarbon, with an alkaline metal base;
f. recovering duloxetine-base;
g. combining the duloxetine-base and a solvent selected from the group consisting of an aromatic hydrocarbon, a C₁₋₄ ester, which is not ethyl acetate, a C₂₋₈ ether, a C₁₋₈ alcohol, acetonitrile and a ketone;
h. adding hydrochloric acid in an amount sufficient to provide a pH of about 1 to about 5 to obtain duloxetine hydrochloride; and
i. recovering duloxetine hydrochloride.

48. The process of claim 47, wherein the DNT-Oxal is (S)-(+)-DNT-Oxal, the DNT-base is (S)-DNT-base, the duloxetine alkyl carbamate is an (S)-duloxetine alkyl carbamate, the duloxetine-base is (S)-duloxetine-base, and the duloxetine hydrochloride is (S)-(+)-duloxetine hydrochloride.

49. A process for preparing duloxetine hydrochloride comprising:
a. combining DNT-Oxal, water, an ammonium hydroxide solution, and an organic solvent to obtain an organic solution, containing DNT-base;
b. combining the DNT-base, a second organic solvent and a proton trap;
c. adding an alkyl chloroformate or a halo alkyl chloroformate (which is not chloroalkyl chloroformate);
d. recovering the duloxetine alkyl carbamate;
e. combining the duloxetine alkyl carbamate and an organic solvent selected from the group consisting of an aliphatic alcohol, ether and an aromatic hydrocarbon, with an alkaline metal base;
f. recovering duloxetine-base;
g. combining the duloxetine-base and a solvent selected from the group consisting of an aromatic hydrocarbon, a C₁₋₄ ester, which is not ethyl acetate, a C₂₋₈ ether, a C₁₋₈ alcohol, acetonitrile and a ketone;
h. adding hydrochloric acid in an amount sufficient to provide a pH of about 1 to about 5 to obtain duloxetine hydrochloride; and
i. recovering duloxetine hydrochloride.

50. The process of claim 49, wherein the DNT-Oxal is (S)-(+)-DNT-Oxal, the DNT-base is (S)-DNT-base, the duloxetine alkyl carbamate is an (S)-duloxetine alkyl carbamate, the duloxetine-base is (S)-duloxetine-base, and the duloxetine hydrochloride is (S)-(+)-duloxetine hydrochloride.
